(19) United States
(12) Patent Application Publication
(10) Pub. No.: US 2018/0298391 A1
(43) Pub. Date: Oct. 18, 2018

(54) Programmable Modification of DNA

(71) Applicant: Massachusetts Institute of Technology, Cambridge, MA (US)

(72) Inventors: Noah Jakimo, Boston, MA (US); Peter A. Carr, Medford, MA (US); Joseph M. Jacobson, Newton, MA (US)

(73) Assignee: Massachusetts Institute of Technology, Cambridge, MA (US)

(21) Appl. No.: 15/905,817

(22) Filed: Feb. 26, 2018

Related U.S. Application Data
(63) Continuation of application No. 14/217,426, filed on Mar. 17, 2014, now abandoned.
(60) Provisional application No. 61/789,524, filed on Mar. 15, 2013.

(51) Int. Cl. C12N 15/63; C12N 15/10

(52) U.S. Cl. CPC C12N 15/635; C12N 15/102

(57) ABSTRACT

A self-reconfiguring genome uses a cassette having operons or DNA sequences that code for guide RNA, reverse transcriptase, donor RNA, and a CRISPR cleavage enzyme. A self-reconfiguring genome may be based on lambda recombineering of in situ generated oligonucleotides. A method for programmable self-modification of a cellular genome includes transcribing guide RNA from a self-reconfiguring cassette, associating the transcribed guideRNA with the CRISPR enzyme, intercalcating a region of complimentary sequence within an integration site of the genome, cutting upstream of a PAM site within the integration site; transcribing the donorRNA, translating donorRNA to double-stranded DNA, and recombining the double-stranded DNA via homologous recombination at the cut site of the integration site. A set of cascadable and multiplexable genetic logic gates with a universal RNA input/output based on single-strand annealing or non-homologous end joining, comprises transcription promoters or terminators, homologous regions, DNA sequences, RNA, and enzymes from the CRISPR system.

### RELATED APPLICATIONS

This application is a continuation of U.S. patent application Ser. No. 14/217,426, filed Mar. 17, 2014, which claims the benefit of U.S. Provisional Application Ser. No. 61/789,524, filed Mar. 15, 2013, the entire disclosures of which are herein incorporated by reference.

### FIELD OF THE TECHNOLOGY

The present invention relates to synthetic biology and, in particular, to methods for programmable modification of DNA.

### BACKGROUND

There is significant current interest in the field of Synthetic Biology, which is a genetic engineering discipline that aims to realize the tools and technologies required for programming biological organisms to perform new functions that they did not previously perform, a task that is somewhat analogous to programming a microprocessor to carry out a new function.

Currently in synthetic biology, exogenous DNA constructs (e.g., genes) are introduced into a biological cell by a number of possible means, including electroporation, opto-poration, chemical competency, conjugation, and viral packaging. These exogenous DNA constructs may then be incorporated into the biological cell's genome, or they may remain as a separate entity within the cell (e.g., as a plasmid). In turn, they may be transcribed into mRNA by the cell's RNA polymerase, which in turn may itself be translated into protein by the cells ribosomal machinery. The exogenous DNA, which codes for novel protein functionality, may ultimately result in programming the cell to carry out a range of new functions, including the incorporation of new exogenous genes that code for the expression of a protein of interest (e.g., protein drugs such as EPO or enzymes such as Amylase), for the incorporation of new exogenous genes that comprise metabolic pathways to program the cell to make a set of new enzymes that in turn synthesize a new compound of interest (e.g., 1,3 Propanediol, Artimisinin), or for the incorporation of sets of genes to perform logic functions (e.g., a ring oscillator causing the cell to blink on and off).

Clustered Regularly Interspaced Short Palindromic Repeats (CRISPRs) have previously been used in a system for programmable double stranded cutting of an integration site [Mali, Prashant, et al., “RNA-guided human genome engineering via Cas9”, Science 339.6121 pp. 823-826 (2013)]. FIG. 1 illustrates the prior art CRISPR—Cas9 system (SEQ ID No. 1, SEQ ID No. 3). Shown in FIG. 1 are integration site 110, guide RNA 120 (SEQ ID No. 2), cleavage sites 130, PAM 140, and Cas 9 protein 150.

A key missing component of synthetic biology as it currently exists is a means for the cell to programmatically modify its own DNA or genome, which is akin to a program rewriting its own memory (e.g., a Turing machine). Applications of this would include cells that can log data, cells that can carry out logic operations, and self-reconfiguring genomes for synthetic evolution and genomic engineering.

Layered logic in engineered genetic circuits is another longstanding goal of synthetic biology. Recent attempts have fallen short due to the difficulty of mining or applying directed evolution to find non-interacting recombinases or pairs of chaperone and transcription factor proteins.

FIGS. 2A-C depicts prior art examples of transcription factor-based logic. In FIG. 2A, two genetic “AND” gates 210, 220 input into third “AND” gate 230. Inputs 240, 245, 250, 255 to the first layer of gates are pairs of chaperone 240, 245 and transcription factor 250, 255 proteins, expressed by inducible promoter. One gate 210 in the first layer outputs chaperone and the other gate 220 outputs a transcription factor, which serve as the input to the second layer gate 230 that outputs RFP 270, as described in T. S. Moon, C. Lou, A. Tamsir, B. C. Stanton and C. A. Voigt, Nature 491, pp. 249-253 (2012). FIGS. 2B and 2C are graphs of output promoter activity (FIG. 2B) and count vs. fluorescence (FIG. 2C) for the transcription factor-based logic gate of FIG. 2A.

FIG. 3 depicts prior art examples of recombinase based logic. Shown in FIG. 3 is a complete set of two-input-one-output logic based on flanking transcription promoters or terminators with Bxb1 and phiC31 recombinase flip sites, as described in Siuti, P., Yazbek, J. & Lu, T. K., “Synthetic circuits integrating logic and memory in living cells”, Nature Biotech, 10 Feb 2013 (doi: 10.1038/nbt.2510).

FIG. 4 illustrates the prior art process of directed nuclease assisted homologous recombination upon cleavage targeted by Zinc fingers, TALs, or Cas9-RNA complex, as described in Esvelt K. M., Wang H. W., “Genome-scale engineering for systems and synthetic biology”, Mol Syst Biol 9: 641, (2013). Shown in FIG. 4 are directed nucleases 410, zinc fingers 420, Cas9 430, crRNA 440, TALs 450, Target 460, Donor 470 with homologous arms 475, and resulting modified genome 480.

FIG. 5 illustrates the prior art process of deletion by single-strand annealing (SSA) homologous recombination. In FIG. 5, double strand break in DNA 510 results in 5′ to 3′ resection 520. Bold complementary regions hybridize 530 when they are both resected. Unpaired single stranded 3′ ends are then removed and the resulting DNA is ligated 540, as described in Frankenberg-Schwager M, Gebauer A, Koppe C, Wolf H, Pralle E, Frankenberg D., “Single-strand annealing, conservative homologous recombination, nonhomologous DNA end joining, and the cell cycle-dependent repair of DNA double-strand breaks induced by sparsely or densely ionizing radiation”, Radiat Res 171, pp. 265-73 (2009).

### SUMMARY

The present invention is a methodology that provides the means for a biological cell to programmatically modify its own DNA. The invention is also self-reconfiguring genomes capable of carrying out the methodology of the invention in order to programmatically modify their own DNA. Applications include, but are not limited to, cells that can log data, cells that can carry out logic operations, and self-reconfiguring genomes for synthetic evolution and genomic engineering. The present invention is also a methodology providing the means for a biological cell to carry out cascadable and multiplexable digital logic using RNA as a universal input and output, a set of genetic logic gates usable in carrying out the methodology, and devices created using the set of genetic logic gates.

In one aspect of the invention, a self-reconfiguring genome is based on a self-reconfiguring cassette that comprises operons or DNA sequences that code for a guide RNA, a reverse transcriptase, donor RNA, and a cleavage enzyme from the CRISPR system. The self-reconfiguring genome may be configured to comprise a counter or data logger, which may be configured to log the presence of a small molecule, peptide, protein, DNA, RNA, heat, and/or light. The self-reconfiguring genome may be configured to reconfigure one or more of an organism's metabolic pathways.

In another aspect of the invention, a self-reconfiguring genome is based on lambda recombineering of in situ generated oligonucleotides. The self-reconfiguring genome based on lambda recombineering may be configured to reconfigure one or more of an organism's metabolic pathways. The self-reconfiguring genome based on lambda recombineering may be configured to comprise a data logger, which may be configured to log the presence of a small molecule, peptide, protein, DNA, RNA, heat, and/or light. The self-reconfiguring genome based on lambda recombineering may be configured so that in situ generated oligonucleotides are generated by means of in situ reverse transcription of RNA.

In a further aspect of the invention, a method for programmable self-modification of a cellular genome includes the steps of, for a self-reconfiguring cassette comprising operons or DNA sequences that code for a guide RNA, a reverse transcriptase, donor RNA, and a cleavage enzyme from the CRISPR system: transcribing the guide RNA from the cassette; associating the transcribed guideRNA with the CRISPR enzyme; intercalcating a region of complimentary sequence within an integration site of the cellular genome; cutting, using the CRISPR enzyme, upstream of a PAM site located within the integration site; transcribing the donor RNA from the cassette; translating the donorRNA to double-stranded DNA using the reverse transcriptase; and recombining the double-stranded DNA via homologous recombination at the cut site of the integration site, thereby producing a genomic modification within the integration site of the cellular genome. The steps of the method may be repeated a plurality of times in order to create serial insertions at the integration site, thereby producing further modification of the cellular genome.

In yet another aspect of the invention, a set of cascadable and multiplexable genetic logic gates with a universal RNA input/output based on single-strand annealing or non-homologous end joining, comprises transcription promoters or terminators, homologous regions, DNA sequences, RNA, and enzymes from the CRISPR system. A genetic logic device may be made of a plurality of genetic logic gates from the set. In the logic device, the genetic logic gates may be cascaded or multiplexed.

### BRIEF DESCRIPTION OF THE DRAWINGS

Other aspects, advantages and novel features of the invention will become more apparent from the following detailed description of the invention when considered in conjunction with the accompanying drawings, wherein:

FIG. 1 illustrates the prior art CRISPR—Cas9 system (SEQ ID No. 1, SEQ ID No. 2, SEQ ID No. 3) for programmable double stranded cutting of an integration site;

FIGS. 2A-C depict prior art examples of transcription factor based logic;

FIG. 3 depicts prior art examples of recombinase based logic;

FIG. 4 illustrates the prior art process of directed nuclease assisted homologous recombination;

FIG. 5 illustrates the prior art process of deletion by single-strand annealing (SSA) homologous recombination;

FIGS. 6A-C together provide a schematic drawing of an exemplary embodiment of a self-reconfiguring genetic cassette (SEQ ID Nos. 4-11) according to one aspect of the invention;

FIGS. 7A-H together provide a schematic drawing of an exemplary embodiment of the generation of double stranded DNA donors from mRNA (SEQ ID Nos. 12-15) according to one aspect of the invention;

FIGS. 8 (SEQ ID Nos. 16-18) and 9 (SEQ ID Nos. 19-22) are schematic drawings of parts of an exemplary embodiment of a counter or data logger that adds segments of DNA to the genome as a function of time or stimulus, according to one aspect of the invention;

FIG. 10 illustrates an exemplary embodiment of a self-reconfiguring system based on lambda recombination, according to one aspect of the invention;

FIG. 11 is illustrates an alternate embodiment of a self-reconfiguring system based on lambda recombination, according to one aspect of the invention;

FIG. 12 is a schematic drawing of an exemplary embodiment of genetic logic gates that cascade, according to one aspect of the invention;

FIG. 13 is a schematic drawing of an exemplary embodiment of genetic logic gates that multiplex, according to one aspect of the invention;

FIG. 14 is a schematic drawing of an exemplary embodiment of alternative genetic logic gates that cascade, according to one aspect of the invention;

FIG. 15 depicts the sequence (SEQ ID No. 23) resulting from experimentally cloning a reporter with the T7 promoter followed by the first 171 bases of GFP, a protospacer and protospacer adjacent sequence, transcription terminator, and the entire GFP gene into BL21 E. coli; and

FIG. 16 depicts an experimentally produced sequence (SEQ ID No. 24) consistent with SSA repair, resulting from introducing the corresponding guide RNA and Cas9 to the sequence (SEQ ID No. 23) of FIG. 15.

### DETAILED DESCRIPTION

In some embodiments, means based on Clustered Regularly Interspaced Short Palindromic Repeats (CRISPRs) allow the cell to self-reconfigure its own genome. A self-reconfiguring cassette according to one aspect of the invention comprises operons or DNA sequences which code for i) a guide RNA to recognize and cleave at an integration site, ii) the CRISPR protein Cas9, iii) reverse transcriptase, and iv) Donor RNA, which is reverse transcribed into double stranded donor DNA.

In some embodiments, the cassette operates in the following manner. Guide RNA (guideRNA) is transcribed from the cassette, associates with the protein CAS9 and intercalates a region of complimentary sequence within the Integration site. Once intercalated, the Cas9 cuts upstream of a PAM site also located within the Integration site. In parallel, donor RNA, whose termini are homologous to the integration site cut site, is transcribed from the cassette by RNA polymerase and then translated to double stranded DNA by means of reverse transcriptase. The double stranded DNA is recombined via homologous recombination at the integration site cut site to produce a genomic modification within the integration site. This serves as a general means for the cell to modify its own genome.

Serial insertions at the integration site can act as a counter. Serial insertions triggered by a stimuli, such as, but not limited to, light small molecular protein, or RNA/DNA, comprise a data logger. Structuring guide RNA sequences and donor DNAs to target promoters or ribosome binding sites within metabolic pathways may comprise a system for carrying out synthetic evolution, diversity or library generation and genomic engineering.

In some other embodiments, means based on CRISPRs allow the cell to carry out cascadable and multiplexable digital logic. In such embodiments, input RNA combines with the Cas9 protein to cut a protospacer sequence, complementary to a spacer sequence in the RNA, followed by a PAM sequence in DNA of the genetic logic gate. This DNA break results in deletion of a transcription promoter or terminator by means of single-strand annealing (SSA) homologous recombination or non-homologous end joining (NHEJ). Output RNA either self-cleaves or is cleaved by Csy4 at CRISPR repeat sequences to improve its affinity for Cas9, thus serving as input for the next layer of gates. The sequence space of such RNA prevents interaction between gates.

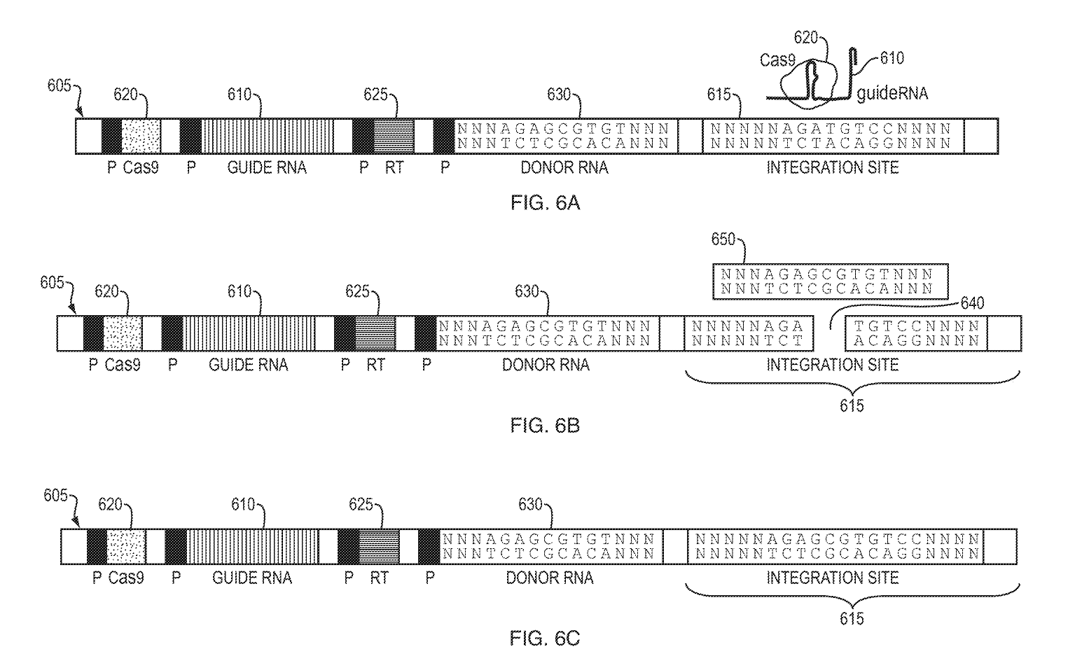

FIGS. 6A-C together provide a schematic drawing of an exemplary embodiment of a self-reconfiguring genetic cassette according to one aspect of the invention. Referring to FIG. 6A, a self-reconfiguring DNA cassette 605 based on Clustered Regularly Interspaced Short Palindromic Repeats (CRISPRs) comprises operons or DNA sequences which code for i) a guide RNA 610 (SEQ ID No. 4, SEQ ID No. 5) to recognize and cleave at an integration site 615 (SEQ ID No. 6, SEQ ID No. 7), ii) the CRISPR protein Cas9 620, iii) reverse transcriptase 625, and iv) Donor RNA 630 which is reverse transcribed into double stranded donor DNA. Guide RNA 610 (guideRNA) is transcribed from cassette 605, associates with the protein Cas9 620 and intercalates a region of complimentary sequence within Integration site 615.

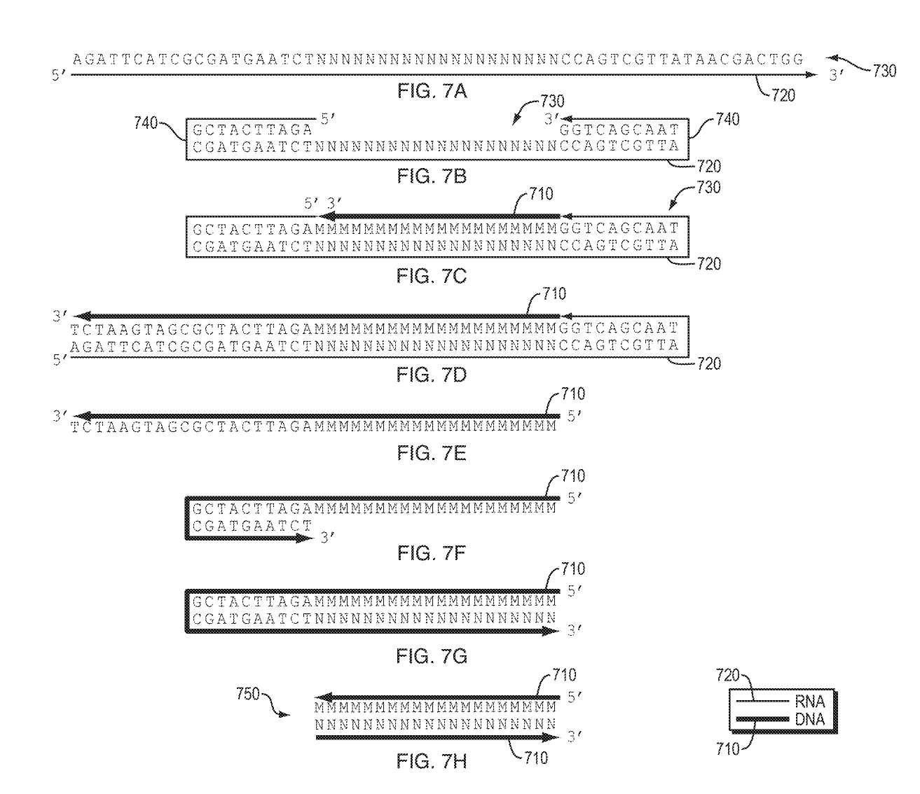

Referring to FIG. 6B, once intercalated, the Cas9 620 cuts upstream of a Proto-spacer Adjacent Motif (PAM) site 640 also located within integration site 615. In parallel, donor RNA 630 whose termini are homologous to the integration site cut site, is transcribed from the cassette by RNA polymerase and then translated to double stranded donor DNA 650 (SEQ ID No. 8, SEQ ID No. 9) by means of reverse transcriptase 620. This reverse transcription may take place by the normal mechanism of reverse transcription employed by retroviruses, which leaves over-flanking heterologous (non-homologous) sequence or by a novel approach, depicted in FIGS. 7A-H, which can generate double stranded donor DNA without heterologous flanking sequence.

Referring to FIG. 6C, double stranded donor DNA 650 is recombined via the cell's homologous recombination system at integration site cut site 640 to produce a DNA sequence modification (SEQ ID No. 10, SEQ ID No. 11) within integration site 615. Such homologous recombination efficiency in bacteria is greatly enhanced by engineering the λ prophage Red recombination system [Zhang, Yongwei, Uwe Werling, and Winfried Edelmann, “SLiCE: a novel bacterial cell extract-based DNA cloning method”, Nucleic Acids Research 40.8, pp. e55-e55 (2012)]. In the strain termed PPY, such homologous recombination can take place at high efficiency, either without heterologous flanking sequence or with short (<˜45 bp) heterologous flanking sequence, although the efficiency is greater without appreciable heterologous flanking sequence.

FIGS. 7A-H together outline the steps for an exemplary embodiment of the generation of double stranded DNA donors from mRNA transcripts according to one aspect of the invention. In FIGS. 7A-H, darker lines 710 represents DNA and lighter lines 720 represent RNA. FIG. 7A depicts an mRNA transcript 730 (SEQ ID No. 12) designed to be self-priming by including hairpin sequences at both the 3′ and 5′ ends. FIG. 7B depicts the mRNA 730 having formed hairpins 740 at both the 3′ end and 5′ end. FIG. 7C (SEQ ID No. 13) depicts Reverse Transcriptase transcribing the mRNA 730 into DNA in the 3′ to 5′ direction. FIG. 7D (SEQ ID No. 14) depicts Reverse Transcriptase displacement of the 5′ end mRNA hairpin and continuation of the DNA transcript in the 3′ direction. FIG. 7E depicts digestion of the mRNA by an RNAse which may be the native RNAse activity of reverse transcriptase. FIG. 7F depicts hairpinning and self-priming of the DNA transcript. FIG. 7G depicts extension of the DNA transcript by DNA polymerase or the DNA polymerase activity of Reverse Transcriptase. FIG. 7G (SEQ ID No. 15) depicts optional restriction enzyme cleavage of the hairpin region of the DNA transcript producing a clean double stranded donor DNA 750.

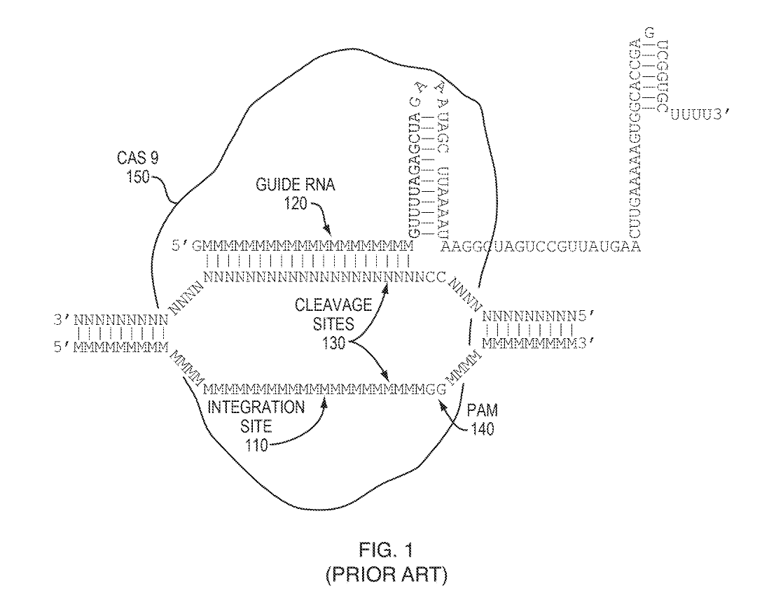

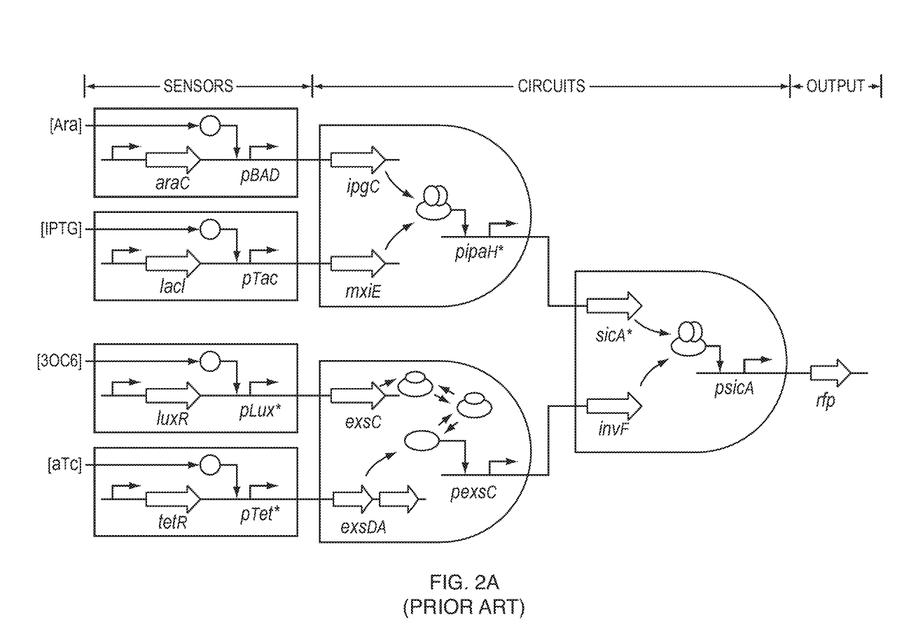

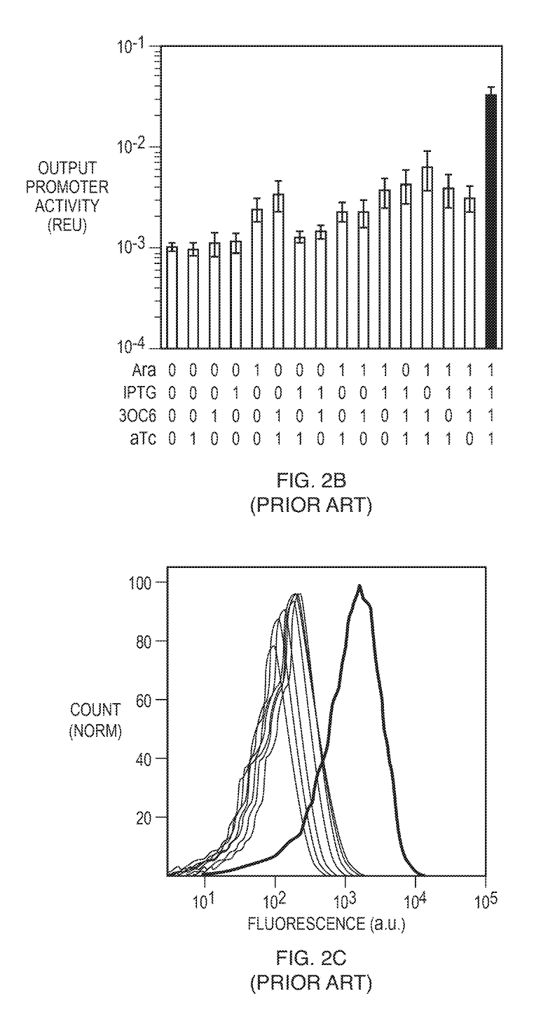

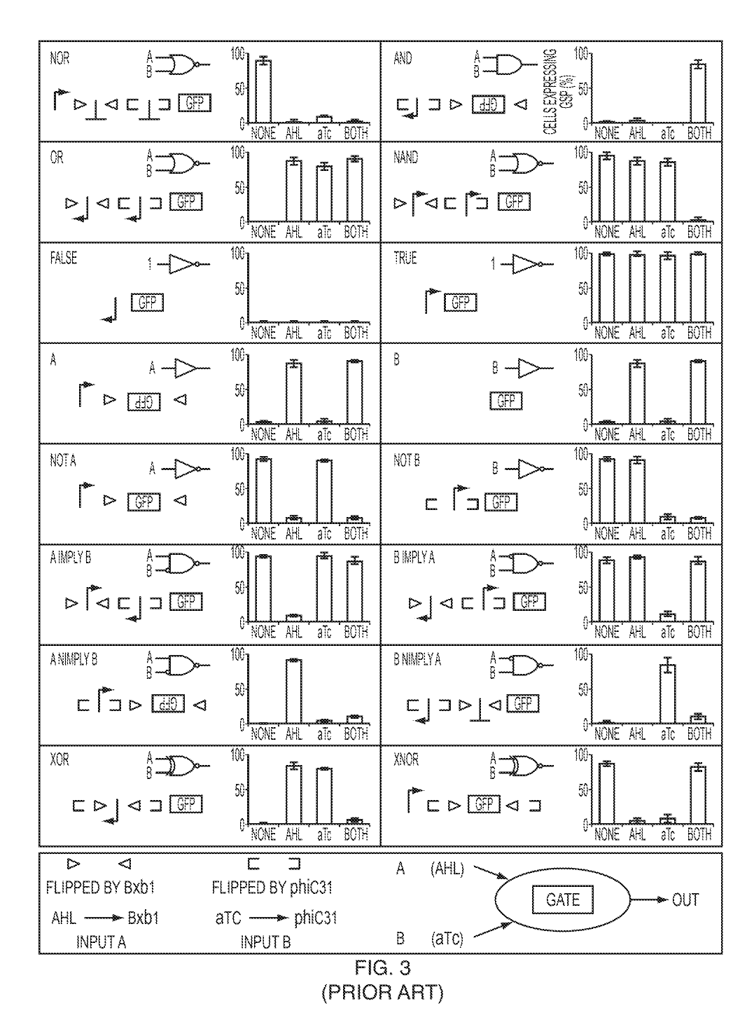

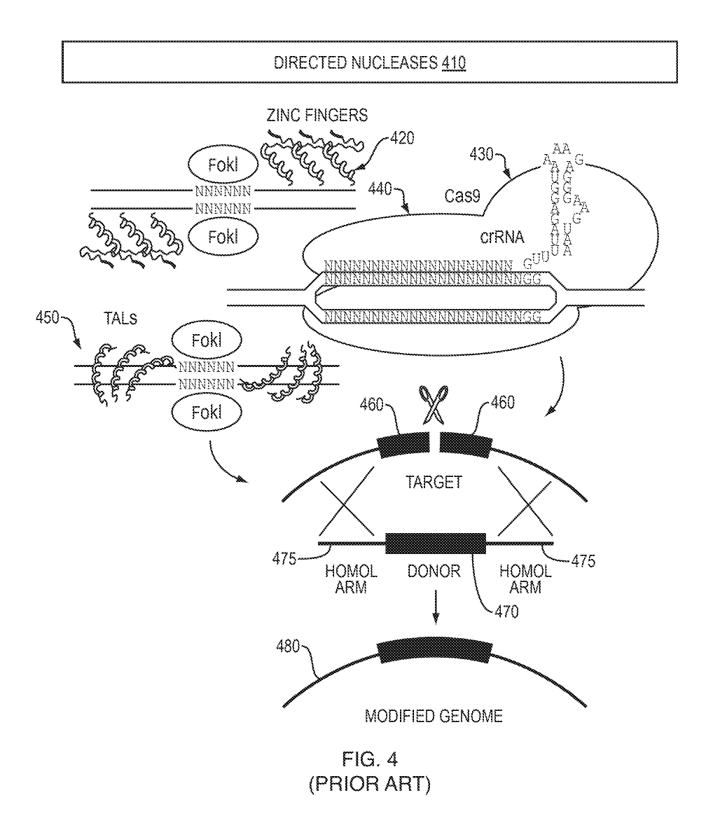

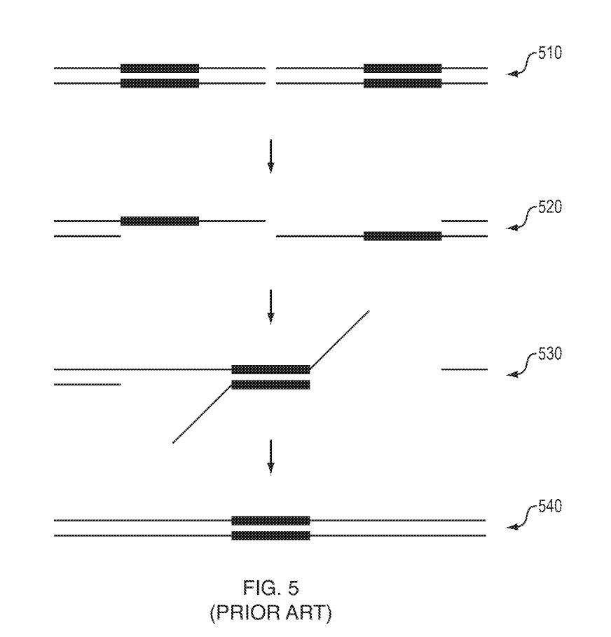

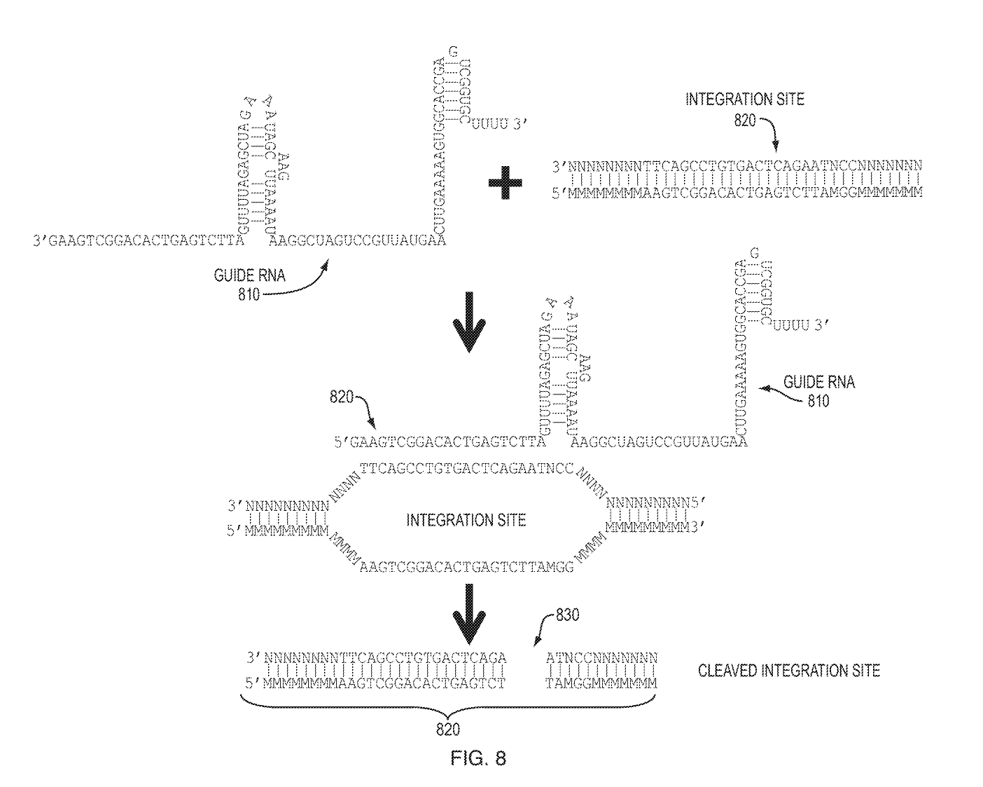

FIGS. 8 and 9 are schematic drawings of parts of an exemplary embodiment of a counter or data logger that adds segments of DNA to the genome as a function of time or stimulus. These added segments may be read out by sequencing of the resultant modified genome. Referring to FIG. 8, a guide RNA 810 (SEQ ID No. 16) which targets integration site 820 (SEQ ID No. 17, SEQ ID No. 18) is expressed either as a function of time or as a function of an input stimulus (e.g., a small molecule such a tetracycline) that activates the promoter for the guide RNA 810. As described previously with respect to FIGS. 1 and 6A-C, the guide RNA 810 complexes with Cas 9 and induces a double stranded break 830 near the PAM sequence of the integration site 820.

Referring to FIG. 9, as discussed with respect to FIGS. 6A-C, double stranded (ds) donor DNA 910 (SEQ ID No. 19, SEQ ID No. 20) can now template the repair of the ds break 830 and add additional DNA sequence 920 to cleaved integration site 820, thus producing modified integration site 930 (SEQ ID No. 21, SEQ ID No. 22) and recording a stimulus event or the passage of time. This process may be continued by having a second guide RNA that now targets and cleaves the newly modified integration site near its PAM site and a second ds donor DNA which templates the repair of that new break and adds additional genetic sequence. If it is arranged that the second ds donor DNA has the same sequence as the original integration site, then this process will circle back on itself with the first guide RNA now targeting the integration site again and so on.

Designing guide RNA sequences and donor DNAs to target promoters or ribosome binding sites within metabolic pathways comprises a system for carrying out self-evolution, diversity or library generation, and self-genomic engineering analogous to the evolution, library generation, and genomic engineering carried out in the process known as MAGE, using exogenously introduced oligonucleotides [Wang, Harris H., et al., “Programming cells by multiplex genome engineering and accelerated evolution”, Nature 460.7257, pp. 894-898 (2009)].

Lambda phage protein (red locus) mediated recombineering can be used to incorporate exogenous oligonucleotides into a chromosome, a form of in vivo site-directed mutagenesis [D. Court et. al., “Genetic Engineering Using Homologous Recombination”, Annual Review of Genetics, Vol. 36, p. 361 (2002)]. The efficiency of this process can be high enough that antibiotic selection is unnecessary, as one can simply screen for recombinants. However, when multiple exogenous oligos are introduced into the cell simultaneously, such as by electroporation or chemical competency, the efficiency of incorporation of each oligo decreases substantially. One limiting factor can be the supply of available β protein. Another can be the amount of each oligo available in the cell. To remedy the second concern, the production of oligos intracellularly, from a plasmid template, is employed. The large plasmid (or BAC) is produced in vivo using gene synthesis techniques, and then transformed into the host. The plasmid is then induced to manufacture large numbers of each desired oligo, which in turn self-reconfigures the genome of the cell.

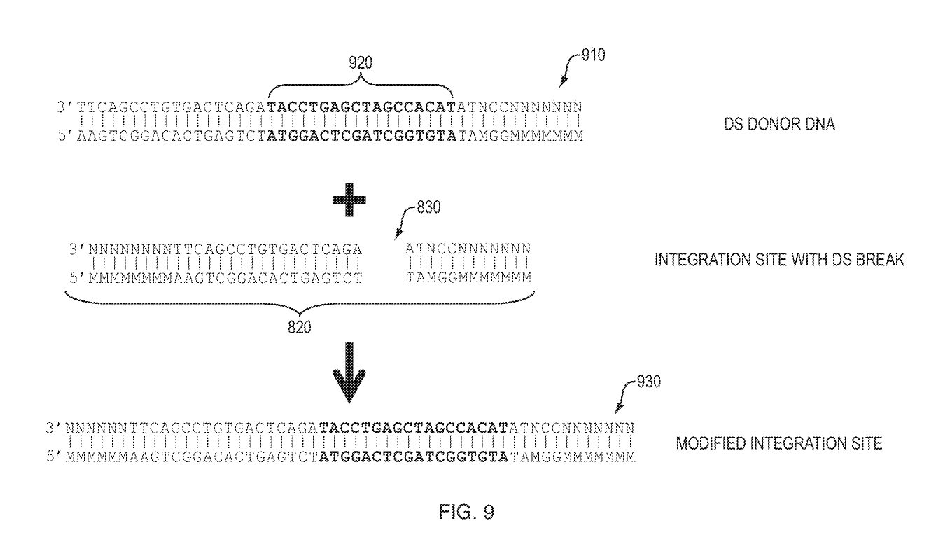

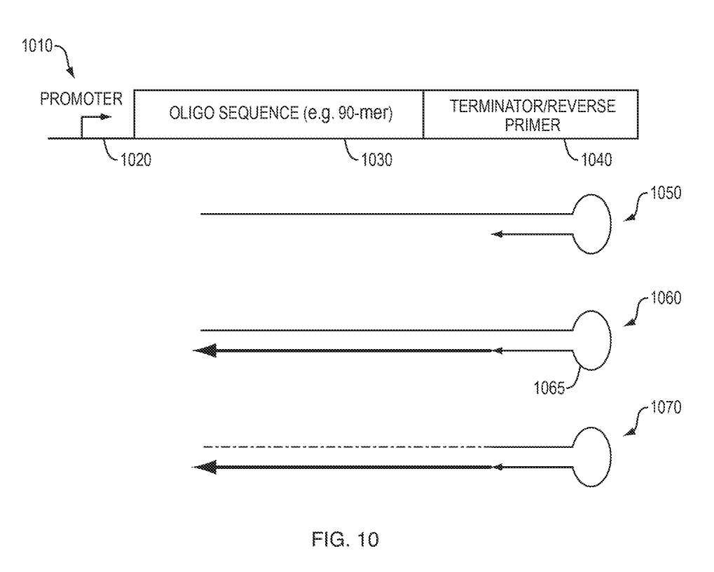

FIGS. 10 and 11 illustrate exemplary embodiments of a self-reconfiguring system based on lamda recombination, according to one aspect of the invention. Referring to FIG. 10, a DNA cassette 1010 is incorporated into the cell. DNA cassette 1010 comprises an RNA polymerase promoter 1020, a first oligonucleotide sequence 1030, a terminator/reverse primer 1040, and then a second oligonucleotide sequence. Additional oligonucleotide sequences may be incorporated, each separated by a terminator/reverse primer, such as shown in cassette 1110 in FIG. 11, so that the oligonucleotide sequence-terminator/reverse primer element is used repeatedly, there being one per oligo being produced. The oligonucleotides are designed to form a hairpin. The oligonucleotides are transcribed 1050 into RNA by RNA polymerase. Additionally, the cassette codes for reverse transcriptase, which makes 1060 a complimentary DNA strand primed by the RNA hairpin 1065 or by tRNA. Finally, RNAseH activity digests 1070 the RNA strand, yielding single stranded DNA oligonucleotides which are further incorporated into the host genome via lambda mediated recombineering [D. Court et. al., “Genetic Engineering Using Homologous Recombination”, Annual Review of Genetics, Vol. 36, p. 361 (2002)]. If the RNA polymerase promoter is activated by a small molecule, light, protein or other stimulus, then this system comprises a data logger in which the new lambda mediated recombineering modification of the genome records the presence of the stimulus.

Referring to FIG. 11, a DNA cassette 1110 is incorporated into the cell. DNA cassette 1110 comprises a rolling circle amplification (RCA) initiation site 1120, a first oligo sequence 1130, a universal separator 1140, and a second oligo sequence 1150. Additional oligonucleotide sequences may be incorporated, each separated by a universal separator 1140. Inside the cell, polymerase transcribes 1150 single stranded copies 1165 of the template, producing ssDNA 1165 by rolling circle (strand displacing) amplification. The universal separators 1140 are designed to form 1170 double stranded hairpins 1175, which in turn are cleaved by a hairpin nuclease, Y flap nuclease, or an exonuclease designed to cut the separator sequence, thus releasing 1180 single stranded DNA oligos 1185 that are further incorporated into the host genome via lambda mediated recombineering

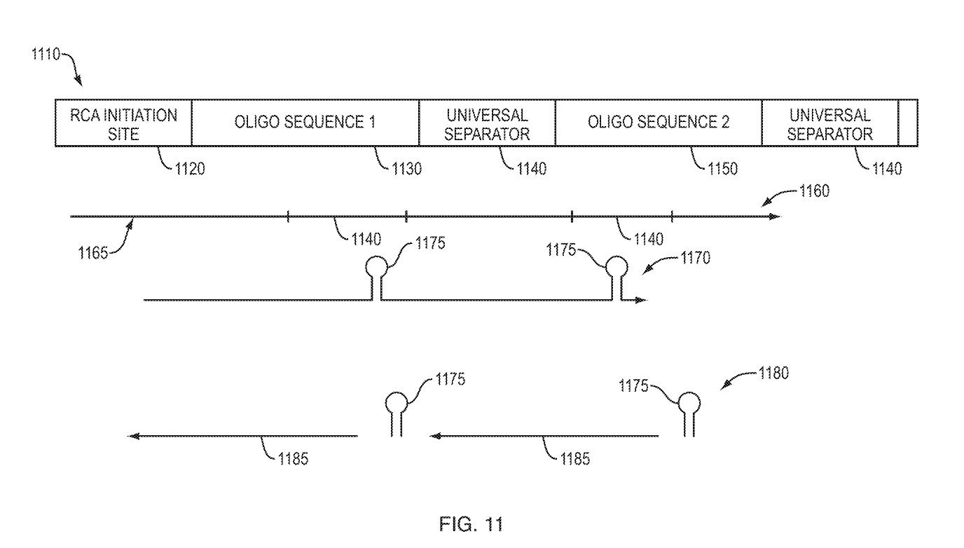

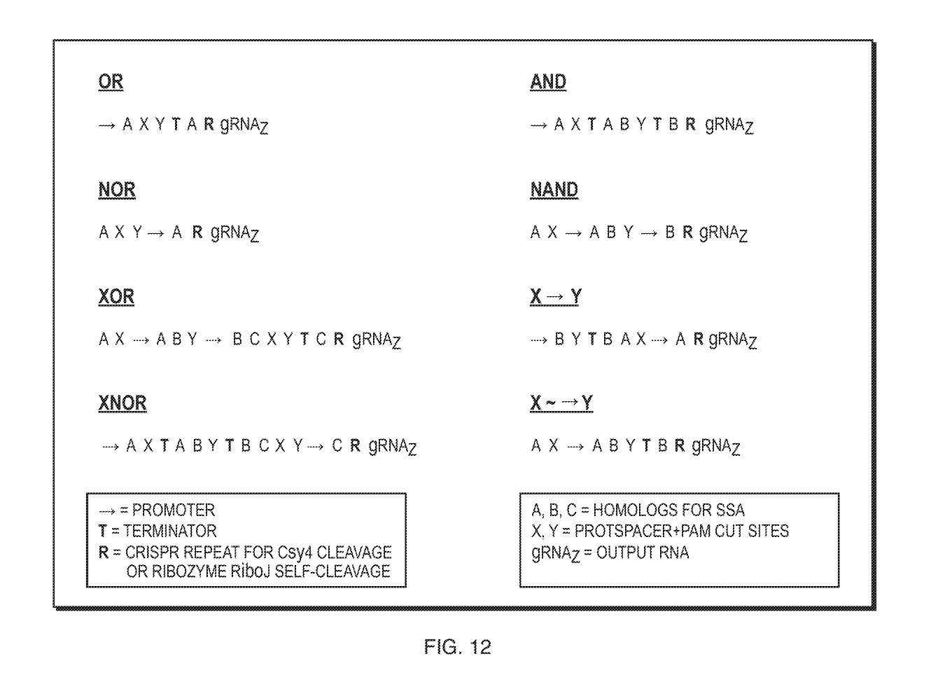

FIG. 12 is a schematic drawing of an exemplary embodiment of genetic logic gates that can be cascaded, according to one aspect of the invention. FIG. 12 depicts all of the non-trivial gates (OR, NOR, XOR, XNOR, AND, NAND, X→Y, and X˜→Y) for a complete set of two-input-one-output logic based on Cas9-gRNA cleavage and SSA homologous recombination. In FIG. 12, “→” represents a promoter, “T” is a terminator, “R” is a CRISPR repeat for Csy4 cleavage or ribozyme RiboJ self-cleavage, “A”, “B”, and “C” are homologs for SSA, “X” and “Y” are protospacer and PAM cut sites, and “gRNA_{z}” represents output RNA. In the system of FIG. 12, gRNA serves as a universal input and output.

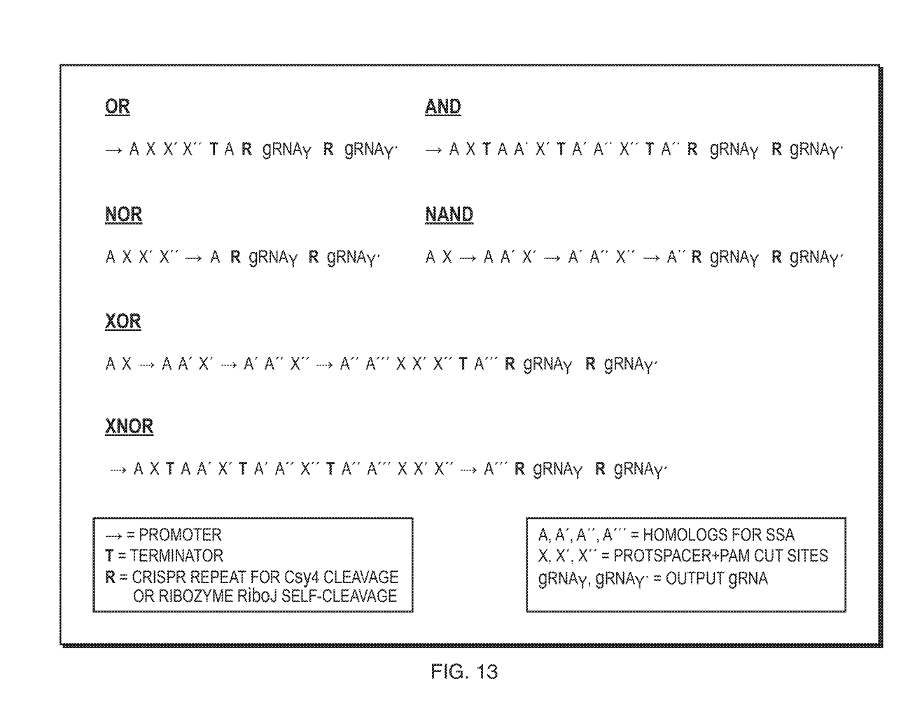

FIG. 13 is a schematic drawing of an exemplary embodiment of three-input-two-output genetic logic gates that multiplex, including OR, NOR, XOR, XNOR, AND, and NAND gates. In FIG. 13, “→” represents a promoter, “T” is a terminator, “R” is a CRISPR repeat for Csy4 cleavage or ribozyme RiboJ self-cleavage, “A”, “A′”, “A″”, and “A′″” are homologs for SSA, “X”, “X′”, and “X″” are protospacer and PAM cut sites, and “gRNA_{Y}” and “gRNA_{Y}” represent output RNA.

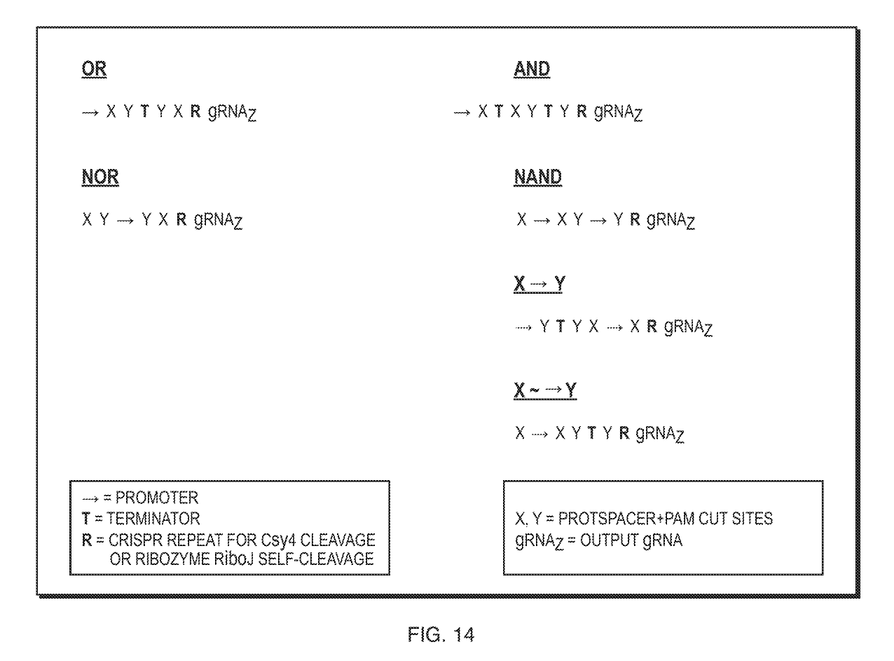

FIG. 14 is a schematic drawing of an exemplary embodiment of alternative genetic logic gates that cascade. FIG. 14 depicts almost all of the non-trivial gates for a complete set of two-input-one-output logic based on Cas9-gRNA cleavage and non-homologous end joining (NHEJ), including OR, NOR, AND, NAND, X→Y, and X˜→Y gates. In FIG. 14, “→” represents a promoter, “T” is a terminator, “R” is a CRISPR repeat for Csy4 cleavage or ribozyme RiboJ self-cleavage, “X” and “Y” are protospacer and PAM cut sites, and “gRNAZ” represents output RNA. In the system of FIG. 14, gRNA serves as a universal input and output.

Logic, universal input/output, and programmable gain are necessary properties for demonstrating computation by single-strand annealing (SSA) homologous recombination repair of CRISPR-induced cleavage. The elements for implementation of this logic have been described above. The parts that make up these elements are well defined: promoter, guide RNA, terminator, RNA processing, and homologous arm sequences.

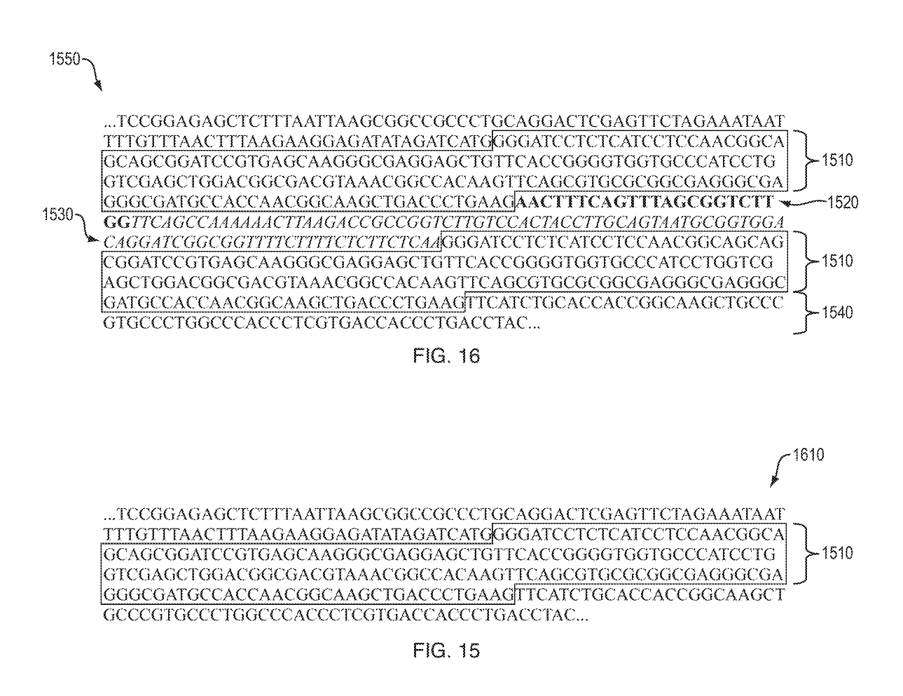

To verify the ideal homologous arm length for instigating SSA, a reporter with the T7 promoter followed by the first 171 bases of GFP 1510 (highlighted), a protospacer and protospacer adjacent sequence 1520 (bold), transcription terminator 1530 (italicized), and the entire GFP gene 1540 were cloned into BL21 E. coli. The resulting construct 1550 (SEQ ID No. 23) is shown in FIG. 15.

Upon introducing the corresponding guide RNA and Cas9, all colonies were found to have sequence 1610 (SEQ ID No. 24) shown in FIG. 16, which is consistent with SSA repair. As hoped, no GFP expression was observed until guide RNA and Cas9 were introduced. To demonstrate universality of input/output and second-layer output, guide RNA will instead follow sequence 1510. In this experiment, second-layer guide RNA targets a sequence on the plasmid to enable quick readout by Surveyor. Gain can then be programmed by adding an array of redundant output guide RNA for increased gain or by adding mismatches to a guide RNA sequence for decreased gain.

Exemplary Implementations: This invention may be implemented in many ways. The items in the list of exemplary implementations that follows are not intended as patent claims. Instead, they are non-limiting examples of ways that this invention may be implemented or embodied. Following are some non-limiting examples of how this invention may be implemented:

Implementation 1. A self-reconfiguring genome based on a self-reconfiguring cassette comprising a guide RNA, a reverse transcriptase, a donor RNA, and a cleavage enzyme from the CRISPR system.

Implementation 2. The system of Implementation 1, configured to comprise a counter.

Implementation 3. The system of Implementation 1, configured to comprise a data logger.

Implementation 4. The system of Implementation 3, configured to comprise a data logger to log the presence of one or more of: small molecule, peptide, protein, DNA, RNA, heat, or light.

Implementation 5. The system of Implementation 1, configured to reconfigure one or more of an organism's metabolic pathways.

Implementation 6. A self-reconfiguring genome based on lambda recombineering of in-situ generated oligonucleotides.

Implementation 7. The system of Implementation 6, configured to reconfigure one or more of an organism's metabolic pathways.

Implementation 8. The system of Implementation 6, configured to comprise a data logger to log the presence of one or more of: small molecule, peptide, protein, DNA, RNA, heat, or light.

Implementation 9. The system of Implementation 6, in which the in situ generated oligonucleotides are generated by means of in situ reverse transcription of RNA.

Implementation 10. Cascadable and multiplexable genetic logic gates with a universal RNA input/output based on single-strand annealing or non-homologous end joining comprising transcription promoters or terminators, homologous regions, as well as DNA sequences, RNA, and enzymes from the CRISPR system.

Implementation 11. The system of Implementation 10, configured to cascade genetic logic gates.

Implementation 12. The system of Implementation 10, configured to multiplex genetic logic gates.

While preferred embodiments of the invention are disclosed herein, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention, which is not to be limited except by the claims.

What is claimed is:

1. A method for programmable modification of a cellular genome, the method comprising the steps of:
programming a genetic cassette to effect a desired genomic modification, the cassette comprising operons or DNA sequences that code for a guide RNA, a reverse transcriptase, donor RNA, and a cleavage enzyme from the CRISPR system, the step of programming comprising modifying and providing the guide RNA and the donor RNA;
introducing the programmed cassette into a cell having a target cellular genome; and
causing expression of the cassette by the cell in order to effect the desired genomic modification, wherein the expression is controlled so that cell is caused to self-modify the target cellular genome by performing the steps of:
transcribing the guide RNA from the cassette;
associating the transcribed guideRNA with the CRISPR enzyme;
intercalcating a region of complimentary sequence within an integration site of the cellular genome;
cutting, using the CRISPR enzyme, upstream of a PAM site located within the integration site;
transcribing the donor RNA from the cassette;
translating the donor RNA to double-stranded DNA using the reverse transcriptase; and
recombining the double-stranded DNA via homologous recombination at the cut site of the integration site, thereby producing the desired genomic modification within the integration site of the target cellular genome.

2. The method of claim 1, further comprising the step of repeating the step of causing expression of the cassette a plurality of times in order to create serial insertions at the integration site, thereby producing further modification of the cellular genome.

3. The method of claim 1, wherein the modified genome is configured to comprise a counter.

4. The method of claim 1, wherein the modified genome is configured to comprise a data logger.

5. The method of claim 4, wherein the data logger is configured to log the presence at least one of: small molecule, peptide, protein, DNA, RNA, heat, or light.

6. The method of claim 1, wherein the modified genome is configured to reconfigure one or more of an organism's metabolic pathways.


SEQUENCE LISTING (24 sequences)

SEQ ID NO: 1 - RNA, 101 nt, Artificial Sequence
  CRISPR - Cas9 system
gmmmmmmmmmmmmmmmmmmmmguuuuagagcuagaaauagcaaguuaaaauaaggcuaguccguuaugaacuugaaaaaguggcaccgagucggugcuuuu

SEQ ID NO: 2 - DNA, 49 bp, Artificial Sequence
  CRISPR - Cas9 system DNA strand 1
  misc_feature (1)..(34): n is a, c, g, or t
  misc_feature (37)..(49): n is a, c, g, or t
nnnnnnnnnnnnnnnnnnnnnnnnnnnnnnnnnnccnnnnnnnnnnnnn

SEQ ID NO: 3 - DNA, 49 bp, Artificial Sequence
  CRISPR - Cas9 system DNA strand 2
mmmmmmmmmmmmmmmmmmmmmmmmmmmmmmmmmmggmmmmmmmmmmmmm

SEQ ID NO: 4 - DNA, 15 bp, Artificial Sequence
  Synthetic
  misc_feature (1)..(3): n is a, c, g, t or u
  misc_feature (13)..(15): n is a, c, g, t or u
nnnagagcgtgtnnn

SEQ ID NO: 5 - DNA, 15 bp, Artificial Sequence
  Synthetic
  misc_feature (1)..(3): n is a, c, g, t or u
  misc_feature (13)..(15): n is a, c, g, t or u
nnntctcgcacannn

SEQ ID NO: 6 - DNA, 17 bp, Artificial Sequence
  Synthetic
  misc_feature (1)..(5): n is a, c, g, or t
  misc_feature (14)..(17): n is a, c, g, or t
nnnnnagatgtccnnnn

SEQ ID NO: 7 - DNA, 17 bp, Artificial Sequence
  Synthetic
  misc_feature (1)..(5): n is a, c, g, or t
  misc_feature (14)..(17): n is a, c, g, or t
nnnnntctacaggnnnn

SEQ ID NO: 8 - DNA, 15 bp, Artificial Sequence
  Synthetic
  misc_feature (1)..(3): n is a, c, g, or t
  misc_feature (13)..(15): n is a, c, g, or t
nnnagagcgtgtnnn

SEQ ID NO: 9 - DNA, 15 bp, Artificial Sequence
  Synthetic
  misc_feature (1)..(3): n is a, c, g, or t
  misc_feature (13)..(15): n is a, c, g, or t
nnntctcgcacannn

SEQ ID NO: 10 - DNA, 20 bp, Artificial Sequence
  Synthetic
  misc_feature (1)..(5): n is a, c, g, or t
  misc_feature (17)..(20): n is a, c, g, or t
nnnnnagagcgtgtccnnnn

SEQ ID NO: 11 - DNA, 20 bp, Artificial Sequence
  Synthetic
  misc_feature (1)..(5): n is a, c, g, or t
  misc_feature (17)..(20): n is a, c, g, or t
nnnnntctcgcacaggnnnn

SEQ ID NO: 12 - DNA, 60 bp, Artificial Sequence
  Synthetic
  misc_feature (21)..(40): n is a, c, g, or t
agattcatcgcgatgaatctnnnnnnnnnnnnnnnnnnnnccagtcgttataacgactgg

SEQ ID NO: 13 - DNA, 80 bp, Artificial Sequence
  Synthetic
  misc_feature (21)..(40): n is a, c, g, or t
agattcatcgcgatgaatctnnnnnnnnnnnnnnnnnnnnccagtcgttataacgactggmmmmmmmmmmmmmmmmmmmm

SEQ ID NO: 14 - DNA, 100 bp, Artificial Sequence
  Fig. 7D
  misc_feature (21)..(40): n is a, c, g, or t
agattcatcgcgatgaatctnnnnnnnnnnnnnnnnnnnnccagtcgttataacgactggmmmmmmmmmmmmmmmmmmmmagattcatcgcgatgaatct

SEQ ID NO: 15 - DNA, 60 bp, Artificial Sequence
  Synthetic
  misc_feature (41)..(60): n is a, c, g, or t
mmmmmmmmmmmmmmmmmmmmagattcatcgcgatgaatctnnnnnnnnnnnnnnnnnnnn

SEQ ID NO: 16 - DNA, 101 bp, Artificial Sequence
  Synthetic
gaagtcggacactgagtcttaguuuuagagcuagaaauagcaaguuaaaauaaggcuaguccguuaugaacuugaaaaaguggcaccgagucggugcuuuu

SEQ ID NO: 17 - DNA, 38 bp, Artificial Sequence
  Synthetic
  misc_feature (1)..(8): n is a, c, g, or t
  misc_feature (29)..(29): n is a, c, g, or t
  misc_feature (32)..(38): n is a, c, g, or t
nnnnnnnnttcagcctgtgactcagaatnccnnnnnnn

SEQ ID NO: 18 - DNA, 38 bp, Artificial Sequence
  Synthetic
mmmmmmmmaagtcggacactgagtcttamggmmmmmmm

SEQ ID NO: 19 - DNA, 48 bp, Artificial Sequence
  Synthetic
  misc_feature (39)..(39): n is a, c, g, or t
  misc_feature (42)..(48): n is a, c, g, or t
ttcagcctgtgactcagatacctgagctagccacatatnccnnnnnnn

SEQ ID NO: 20 - DNA, 48 bp, Artificial Sequence
  Synthetic
aagtcggacactgagtctatggactcgatcggtgtatamggmmmmmmm

SEQ ID NO: 21 - DNA, 54 bp, Artificial Sequence
  Synthetic
  misc_feature (1)..(6): n is a, c, g, or t
  misc_feature (45)..(45): n is a, c, g, or t
  misc_feature (48)..(54): n is a, c, g, or t
nnnnnnttcagcctgtgactcagatacctgagctagccacatatnccnnnnnnn

SEQ ID NO: 22 - DNA, 54 bp, Artificial Sequence
  Synthetic
mmmmmmaagtcggacactgagtctatggactcgatcggtgtatamggmmmmmmm

SEQ ID NO: 23 - DNA, 609 bp, Artificial Sequence
  Synthetic
tccggagagctctttaattaagcggccgccctgcaggactcgagttctagaaataattttgtttaactttaagaaggagatatagatcatggggatcctctcatcctccaacggcagcagcggatccgtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagaactttcagtttagcggtcttggttcagccaaaaaacttaagaccgccggtcttgtccactaccttgcagtaatgcggtggacaggatcggcggttttcttttctcttctcaagggatcctctcatcctccaacggcagcagcggatccgtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctac

SEQ ID NO: 24 - DNA, 325 bp, Artificial Sequence
  Synthetic
tccggagagctctttaattaagcggccgccctgcaggactcgagttctagaaataattttgtttaactttaagaaggagatatagatcatggggatcctctcatcctccaacggcagcagcggatccgtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctac